(19) United States
(12) Patent Application Publication
(10) Pub. No.: US 2009/0082421 A1
(43) Pub. Date: Mar. 26, 2009

(54) Crystalline Form B4 of Atorvastatin Magnesium and a Process Thereof

(75) Inventors: Joy Mathew, Karnataka (IN); Manicka Ramamoorthy Sivakumar, Tamilnadu (IN); Poornaprajna Acharya, Karnataka (IN)

Correspondence Address: HARNESS, DICKEY & PIERCE, P.L.C., P.O. BOX 828, BLOOMFIELD HILLS, MI, 48303, US

(73) Assignee: BIOCON LIMITED, Karnataka (IN)

(21) Appl. No.: 12/293,108

(22) PCT Filed: Jun. 15, 2006
(86) PCT No.: PCT/IN2006/000202

§ 371 Date: Sep. 16, 2008

(30) Foreign Application Priority Data
May 11, 2006 (IN) 837/CHE/2006

(51) Int. Cl. A61K 31/40; C07D 207/34

(52) U.S. Cl. 514/423; 548/537

(57) ABSTRACT

Crystalline polymorphic form B4 of Atorvastatin magnesium of FIG. 1 and processes for its preparation.

### FIELD

The present invention is in relation to crystalline form of atorvastatin magnesium as well as a process for its preparation. The novel form is useful as inhibitors of the enzyme3-hydroxy-3-methylglutaryl-coenzyme A reductase (HMG-CoA reductase).

### BACKGROUND

The present invention relates to crystalline form B4 of atorvastatin magnesium, i. e., [R-(R*, R*)]-2-(4-fluorophenyl)-β,δ-,6-dihydroxy-5-(1-methylethyl)-3-phenyl-4-[(phenylamino)-carbonyl]-IH-pyrrole-heptanoic acid magnesium salt (2:1) (represented with FORMULA I), also known as atorvastatin magnesium, the processes for its preparation and isolation, pharmaceutical compositions which include the form B4, and a pharmaceutically acceptable carrier, and to a method of administering a therapeutic amount of the pharmaceutical composition for the treatment of hyperlipidemia and hypercholesterolemia.

The crystalline form has different properties due to the unique arrangement of molecules in the crystal lattice varying density of packing, and/or by varying hydrogen-bond network. Accordingly, individual crystalline form may be thought of as distinct solids having distinct advantageous and/or disadvantageous and/or physical properties compared to other polymorphic form.

### Objects of the Present Invention

The principal object of the present invention is to prepare a new polymorphic form of atorvastatin magnesium, i.e. crystalline form B4, characterized by X-ray powder diffraction pattern.

Another object of the present invention is to develop a new process for preparation of atorvastatin magnesium form B4.

Yet another object of the present invention is to develop a pharmaceutical composition and dosage form comprising crystalline form B4 of atorvastatin magnesium.

Still another objective of the present invention is to develop a method of treating hyperlipidemia and/or hypercholesteremia with a pharmaceutical composition containing a therapeutically effective amount of crystalline form B4 of atorvastatin magnesium.

### SUMMARY

The present invention relates to a crystalline form B4 of atorvastatin magnesium which is characterized by X-Ray powder diffraction pattern as shown in FIG. 1;

a process for preparation of crystalline form B4 of atorvastatin magnesium as shown in FIG. 1, said process comprising steps of: (a) adding suitable solvent or solvent mixture to atorvastatin magnesium to obtain a mixture; and (b) isolating the crystalline form B4 of atorvastatin magnesium from the mixture;

a pharmaceutical composition comprising crystalline form B4 of atorvastatin magnesium and pharmaceutically acceptable additives;

a method of inhibiting enzyme 3-hydroxy-3-methylglutaryl-coenzyme A reductase (HMG-CoA reductase) in a subject in need thereof, said method comprising step of administering pharmaceutically effective amount of crystalline form B4 of atorvastatin magnesium optionally along with the pharmaceutically acceptable additives to the subject; and

a method of treating or preventing or palliating hypercholesterolemia and/or hyperlipidemia in a subject in need thereof, said method comprising step of administering pharmaceutically effective amount of crystalline form B4 of atorvastatin magnesium optionally along with the pharmaceutically acceptable additives to the subject.

### DETAILED DESCRIPTION

The present invention relates to a crystalline form B4 of atorvastatin magnesium characterized by X-Ray powder diffraction pattern as shown in FIG. 1.

Yet another embodiment of the present invention, wherein said form B4 shows purity greater than 98%.

In still another embodiment of the present invention, wherein the form B4 has 100% purity.

The present invention relates to a process for preparation of crystalline form B4 of atorvastatin magnesium as shown in FIG. 1, said process comprising steps of:

adding suitable solvent or solvent mixture to atorvastatin magnesium to obtain a mixture; and

isolating the crystalline form B4 of atorvastatin magnesium from the mixture.

### BRIEF DESCRIPTION OF THE ACCOMPANYING DRAWINGS

The invention is further described by the following non-limiting examples, which refer to the accompanying FIG. 1 briefly described below.

FIG. 1 is a characteristic X-ray powder diffraction pattern of crystalline form B4 of Atorvastatin magnesium.

Yet another embodiment of the present invention, wherein the mixture is optionally subjected to stirring and is allowed to crystallize, followed by filtering and drying.

In still another embodiment of the present invention, wherein the mixture is optionally subjected to suitable temperature ranging between 25-50 degree centigrade.

In still another embodiment of the present invention, wherein the solvent(s) is selected from a group comprising protic, aprotic, water miscible, water immiscible, polar and non-polar solvents.

In still another embodiment of the present invention, wherein solvent(s) is selected from a group comprising water, acetonitrile, methanol, ethanol, acetone, ethyl acetate, chloroform, isopropyl alcohol, tetra hydro furan, dichloromethane, t-butanol, iso-butanol, carbon tetrachloride, 1,4-dioxan, n-butanol, di-isopropyl ether and di-ethyl ether.

In still another embodiment of the present invention, wherein the atorvastatin magnesium is in amorphous and/or crystalline form.

In still another embodiment of the present invention, wherein the crystalline form B4 has purity greater than 98%.

In still another embodiment of the present invention, wherein the crystalline form B4 has 100% purity.

In still another embodiment of the present invention, wherein the preparation of amorphous form of atorvastatin magnesium comprising steps of;

a. adding methanol and water to atorvastatin magnesium to attain a mixture; and

b. isolating the amorphous form of atorvastatin magnesium.

In still another embodiment of the present invention, wherein the temperature is raised or lowered to afford the crystalline form B4 of atorvastatin magnesium.

The present invention is in relation to a pharmaceutical composition comprising crystalline form B4 of atorvastatin magnesium and pharmaceutically acceptable additives.

Yet another embodiment of the present invention, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

The present invention relates to a method of inhibiting enzyme 3-hydroxy-3-methylglutaryl-coenzyme A reductase (HMG-CoA reductase) in a subject in need thereof, said method comprising step of administering pharmaceutically effective amount of crystalline form B4 of atorvastatin magnesium optionally along with the pharmaceutically acceptable additives to the subject.

The present invention relates to a method of treating or preventing or palliating hypercholesterolemia and/or hyperlipidemia in a subject in need thereof, said method comprising step of administering pharmaceutically effective amount of crystalline form B4 of atorvastatin magnesium optionally along with the pharmaceutically acceptable additives to the subject.

Yet another embodiment of the present invention, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

In still another embodiment of the present invention, wherein the dose of crystalline form B4 of atorvastatin magnesium is ranging between 0.5 to 100 mg.

The present invention relates to Atorvastatin magnesium of purity greater than 98.0%.

The present invention relates to Atorvastatin magnesium of 100% purity.

In still another embodiment of the present invention, it has been invented that atorvastatin magnesium can be prepared in additional crystalline form. Thus, the present invention provides atorvastatin magnesium (2:1) in a new polymorphic form denominated as crystalline form B4. The form B4 exhibit different physical characteristics as is evident from their X-ray powder diffraction patterns.

While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instance, well known process operations have not been described in detail, in order not to obscure the present invention.

This invention is related to crystalline form B4 of [R—(R*, R*)]-2-(4-fluorophenyl)-β,δ-dihydroxy-5-(1-methylethyl)-3-phenyl-4-[(phenylamino)-carbonyl]-IH-pyrrole-heptanoic acid magnesium salt (2:1) having the following generic chemical structure as depicted in Formula I.

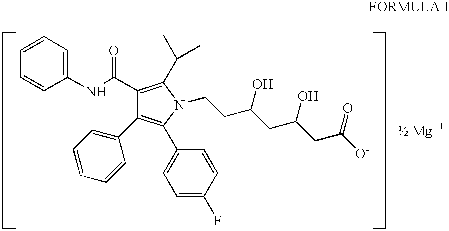

The invention is further directed to the processes for the production and isolation of form B4, to pharmaceutical compositions which include the crystalline form B4, and a pharmaceutically acceptable carrier, and to a method of administering a therapeutic amount of the pharmaceutical composition for the treatment of hyperlipidemia and hypercholesterolemia. The B4 form of atorvastatin magnesium is useful as inhibitors of the enzyme, 3-hydroxy-3-methylglutaryl-coenzyme A reductase, and therefore, are useful as agents for treating hyperlipidemia and hypercholesterolemia.

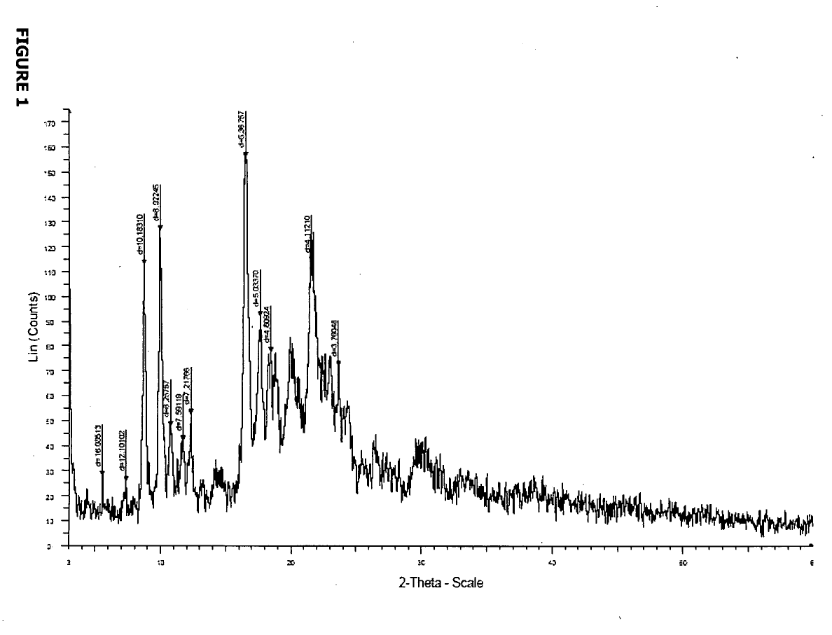

The B4 form is characterized by their distinctive X-ray powder diffractogram, as shown in FIG. 1.

The present invention also provides for a method for the preparation of crystalline form B4 of atorvastatin magnesium (2:1). The method comprises exposing atorvastatin to different solvents and temperature conditions, which yield crystalline form B4.

Crystalline atorvastatin magnesium form B4 may be prepared under controlled conditions. In particular, it can be prepared/isolated by crystallization from aqueous, water-miscible, non-aqueous or non-polar solvents at a suitable temperature. Suitable solvents comprise water, acetonitrile, methanol, ethanol, acetone, ethyl acetate, chloroform, isopropyl alcohol, THF, dichloromethane, t-butanol, iso-butanol, carbon tetrachloride, 1,4-dioxan, n-butanol, di-isopropyl ether or di-ethyl ether.

In one embodiment, atorvastatin magnesium is treated with a mixture of two or more suitable solvents/anti-solvents under a suitable temperature range and the mixture can be then filtered and dried, preferably under vacuum, to obtain crystalline atorvastatin magnesium.

In another embodiment, Atorvastatin magnesium is treated with a suitable solvent or mixture of solvents under a suitable temperature range which can be then dried to obtain amorphous atorvastatin magnesium.

Atorvastatin magnesium in any other crystalline or amorphous form can be directly converted into the form 4 or any other crystalline form can be converted into amorphous form which is then converted into the Form 4.

Preferably, atorvastatin magnesium can be suspended in a solvent or mixture of solvents. Preferably, the solvents are methanol and water. The mixture of solvent/solvents and atorvastatin magnesium can be stirred at suitable temperature and allowed to stand for suitable time to allow crystallization to occur. Preferably, the mixture is allowed to stand for 1-4 days. The mixture can be then filtered and the product was dried at suitable temperature. Preferably the drying is done under vacuum.

The typical XRD of the form 4 of Atorvastatin magnesium is shown in FIG. 1.

The product thus obtained may be of particle size less than 250 micron. The product can be subjected to size reduction or suitable process to attain the desired particle size.

The product obtained by the process can be of pharmaceutically acceptable grade/purity. The product can be obtained with purity more than 98%. Preferably, the product obtained can be of purity more than 99%. The product may be purified to as high as 100% purity by known techniques of extraction, chromatography, crystallization/precipitation etc.

It is expected to find some structurally related or unrelated impurities to be present in the product with varying concentration. The impurities may be present in concentration ranging from 0.001 to 2%. The product containing relatively high amount of impurities may be subjected to suitable processes like, solvent-solvent extraction, chromatography or crystallization to afford product with higher purity.

The impurities that can be found are one or more selected form Atorvastatin Amide, Desfluoro Atorvastatin, O-methyl Atorvastatin, Atorvastatin lactone, Atorvastatin methyl ester, Atorvastatin Di-epoxide, Diketo1,2-Diol, Diketoepoxide atorvastatin lactone, atorvastatin open acid as well as isomers of the open acid, lactone or atorvastatin magnesium. The water content of the product may vary with the API processing conditions, storage conditions, shipment conditions or formulation processing conditions.

It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amounts used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

The compound according to the invention is optionally formulated in a pharmaceutically acceptable vehicle with any of the well known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention, as well any other pharmacologically active ingredient useful for the treatment of the symptom/condition being treated.

The compound of the present invention can be prepared into a pharmaceutical composition by admixing the compound with a pharmaceutically acceptable carrier, adjuvant or vehicle. The resultant pharmaceutical composition can be administered in a wide variety of dosage form, e.g., oral, topical, parenteral or the like. It will be obvious to those skilled in the art that such dosage form, e. g., powders, tablets, pills, capsules, aggregates, suppositories, granules and the like, or liquid form, e.g., solutions, suspensions, or emulsions may comprise as the active component of the present invention. In solid dosage form, the atorvastatin magnesium crystalline form B1 B4 is finely divided or mixed with one or more inactive ingredients, which can act as inactive filling materials, taste or flavor corrigenda, chemical preservatives, solubilizers, lubricants, and the like. In liquid form, the atorvastatin magnesium crystalline form B4 is suspended, emulsified or dissolved in suitable vehicles containing various inactive components, e.g., solvents, buffers, stabilizers, colorants, flavors, and the like. The preferred unit dosages of the pharmaceutical composition of this invention typically contain from 0.5 to 100 mg of atorvastatin magnesium form B4.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

The invention is further elaborated with the help of following example(s). However, these example(s) should not be construed to limit the scope of the invention.

### EXAMPLE 1

To a solution of compound of formula II

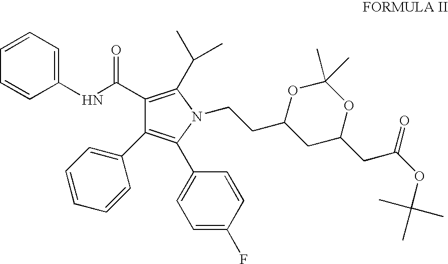

(100 g, 0.153 mol) in methanol (1.8 L), HCl (1 N, 210 mL) was added over a period of 30 minutes and stirred for 2.5 h at ambient temperature. Aqueous solution of sodium hydroxide (10%, 153 mL) was added to the reaction mixture and stirred for 2.5 h at ambient temperature. After completion of reaction (by TLC), pH of the reaction mixture was adjusted to 9.0-9.5 using 1N HCl and the mixture was filtered over celite bed. The filtrate was concentrated to about 400 mL and water (1.0 L) and methyl tert-butyl ether (MTBE, 400 mL) were added. Sufficient quantity of methanol was added to get two layers and MTBE layer was separated. Aqueous layer was further washed with MTBE (400 mL). pH of aqueous layer was adjusted to 7.5-8.0 (using 1N HCl) and washed with MTBE ( 2×400 mL). The aqueous layer was warmed to 40-45° C. and a solution of magnesium acetate tetra-hydrate (24.5 g, 0.114 mol) in water (570 mL) was added over a period of 1 h. After stirring the mixture at 40-45° C. for 15 minutes, it was cooled to about 30° C. over a period of 3 h. Atorvastatin magnesium was filtered and washed with a mixture of water and methanol (in the ratio 8.5:1.5). The product obtained was dissolved in methanol and concentrated under vacuum at temperature 35-45 deg C. to afford atorvastatin magnesium solid of 98.5% purity as measured by HPLC.

### EXAMPLE 2

Compound of formula III

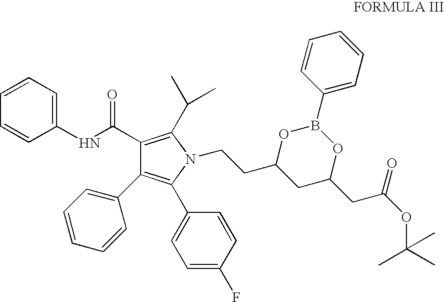

(100 g, 0.142 mol) was suspended in a mixture of methanol (300 mL) and water (1 L) and a solution of sodium hydroxide (28.5 g) in water (90 mL) was added. The mixture was refluxed for 4 h. Reaction mixture was cooled to room temperature and washed with MTBE (400 mL). After separating layers, aqueous layer was kept under vacuum for 1 hour and the solution was allowed to stand for 2 h at room temperature. The precipitate formed was filtered. The product obtained was dissolved in a mixture of water (1 L), methanol (300 mL) and MTBE (400 mL). pH of the aqueous layer was adjusted to 7.5-8.0 with HCl (1N) and MTBE layer separated. The aqueous layer was warmed to 40-45 ° C. and a solution of magnesium acetate tetra-hydrate (22.9 g) in water (75 mL) was added. Reaction mixture was stirred at 40-45° C. for 1 h and cooled to ambient temperature over a period of 1 h. The product was filtered and washed with a mixture of water and methanol (in the ratio 8.5:1.5). The product obtained was dissolved in methanol and concentrated under vacuum at temperature 35-45 deg C. to afford atorvastatin magnesium solid of 99.5% purity as measured by HPLC.

### EXAMPLE 3

Atorvastatin magnesium obtained as in previous examples (100 g) was suspended in a mixture of methanol (500 mL) and water (2 L), stirred at room temperature and allowed to stand upto 3 days. The mixture was filtered and the product was dried under vacuum at 35-50° C. Weight: 80 g. The X-Ray powder diffraction of the product is shown in FIG. 1.

While the salient features have been illustrated and described with respect to particular embodiments, it should be readily apparent that modifications can be made within the spirit and scope of the invention, and it is therefore not desired to limit the invention to the exact details shown and described.

1-21. (canceled)

22. Crystalline form B4 of atorvastatin magnesium characterized by X-Ray powder diffraction pattern comprising 2 theta values 3.02, 5.76, 7.09, 8.65, 9.91, 10.72, 11.51, 12.24, 14.31, 16.44, 17.56, 18.38, 18.75, 20.06, 21.65, 22.90±0.2.

23. The crystalline form B4 according to claim 22, wherein the form shows purity greater than 98%.

24. The crystalline form B 4 according to claim 22, wherein the form has 100% purity.

25. A process for preparation of crystalline form B4 of claim 22, said process comprising steps of:
a. adding a solvent or mixture of solvent to atorvastatin magnesium to obtain a mixture; and
b. isolating the crystalline form B4 of atorvastatin magnesium from the mixture.

26. The process according to claim 25, wherein the mixture is optionally subjected to stirring, allowed to crystallize, followed by filtering and drying.

27. The process according to claim 25, wherein the mixture is optionally subjected to suitable temperature ranging between 25-50° C.

28. The process according to claim 25, wherein the solvent(s) is selected from the group comprising protic, aprotic, water miscible, water immiscible, polar and non-polar solvents.

29. The process according to claim 28, wherein solvent(s) is selected from the group comprising water, acetonitrile, methanol, ethanol, acetone, ethyl acetate, chloroform, isopropyl alcohol, tetra hydro furan, dichloromethane, t-butanol, iso-butanol, carbon tetrachloride, 1,4-dioxan, n-butanol, di-isopropyl ether and di-ethyl ether.

30. The process according to claim 25, wherein the atorvastatin magnesium obtained after step [a] is in amorphous or crystalline form.

31. The process according to claim 25, wherein the crystalline B4 has purity greater than 98%.

32. The process according to claim 25, wherein the crystalline form B4 has 100% purity.

33. The process according to claim 25, wherein the preparation of crystalline form B4 of atorvastatin magnesium comprising steps of;
a. adding methanol and water to atorvastatin magnesium to attain a mixture; and
b. isolating the crystalline form of atorvastatin magnesium.

34. The process according to claim 27, wherein the temperature is raised or lowered to afford the crystalline form B4 of atorvastatin magnesium.

35. A pharmaceutical composition comprising crystalline atorvastatin magnesium in B4 form of claim 22, along with pharmaceutically acceptable additives.

36. The pharmaceutical composition according to claim 35, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

37. A method of inhibiting enzyme 3-hydroxy-3-methylglutaryl-coenzyme A reductase (HMG-CoA reductase) in a subject in need thereof, said method comprising step of administering pharmaceutically effective amount of crystalline atorvastatin magnesium in B4 form of claim 22, along with the pharmaceutically acceptable additives to the subject.

38. A method of treating or preventing or palliating hypercholesterolemia and/ or hyperlipidemia in a subject in need thereof, said method comprising step of administering pharmaceutically effective amount of crystalline atorvastatin magnesium in B4 of claim 22, optionally along with the pharmaceutically acceptable additives to the subject.

39. A method of treatment according to claim 38, wherein the dose of crystalline atorvastatin magnesium B4 is ranging between 0.5 to 100 mg.

40. Atorvastatin magnesium B4 according to claim 22 of purity at least 99.0%.

41. Atorvastatin magnesium B4 according to claim 22 of purity at least 99.5%.
